(19) United States
(12) Patent Application Publication
(10) Pub. No.: US 2026/0071166 A1
(43) Pub. Date: Mar. 12, 2026

(54) System and Method for Perfusion Bioreactor for 3D Cell Culture

(71) Applicant: BMF Precision, Inc., Maynard, MA (US)

(72) Inventor: Chunguang Xia, San Diego, CA (US)

(73) Assignee: BMF Precision, Inc., Maynard, MA (US)

(21) Appl. No.: 19/106,771

(22) PCT Filed: Aug. 25, 2023
(86) PCT No.: PCT/US2023/031125

§ 371 Date: Feb. 26, 2025

Related U.S. Application Data
(60) Provisional application No. 63/401,143, filed on Aug. 26, 2022.

(51) Int. Cl. C12M 1/00; B33Y 80/00; C12M 3/00; C12M 3/06

(52) U.S. Cl. CPC C12M 29/10; C12M 21/08; C12M 23/16; C12M 23/22; C12M 23/34; C12M 23/48; C12M 29/16; B33Y 80/00

(57) ABSTRACT

A 3D perfusion bioreactor system and method for more accurate modeling of in vivo environmental conditions and cellular behaviors. In some embodiments, the bioreactor includes a cavity for containing culture media and cells. A plurality of capillary tubes cross the cavity. Artery capillary tubes may transport culture media received from an inlet of the bioreactor, across the cavity, and into a return compartment. Vein capillary tubes may transport culture media from the return compartment, across the cavity, and to an outlet of the bioreactor. Metabolites may diffuse from the capillary tubes into the cavity for cellular consumption, and metabolic waste may diffuse from the cavity into the capillary tubes for removal from the bioreactor. In some embodiments, culture media discharged from the outlet may be processed via a waste treatment device and returned to the inlet.

### FIELD

This disclosure relates to cell biology and/or tissue engineering, and more particularly, to systems and methods for mimicking a 3D biological environment for cells using a perfusion bioreactor as a 3D cell-culture model.

### BACKGROUND

Recent drug discovery efforts have focused on incorporating in vitro cell models that better mimic the in vivo conditions found within a target patient. Conventionally, a predetermined quantity or concentration of model cells is seeded onto a coated microplate well. The microplate is incubated to encourage the cells to attach in a two-dimensional (2D) monolayer before performing a prescribed assay. While this may provide some improvements over biochemical and immortalized cell lines, culturing cells in this 2D manner may be problematic, at least because 2D cell-culture models typically do not accurately model in vivo environmental conditions and/or cellular behaviors.

For example, studies of 2D cell-culture models have shown that attrition rates of drug candidates for cancer can be as high as approximately 95%. Further, in vitro drug efficacy values can fail to translate to clinical environments. Still further, unforeseen toxicity issues can arise. First, it is believed that in 2D cell-culture models, some extracellular matrix (ECM) components are missing, and therefore, certain cell-to-cell and cell-to-matrix interactions do not occur. These components and interactions are critical to cell differentiation, cell proliferation, and/or cellular function that normally occur in vivo. Second, it has been found that the environment of a 2D cell-culture model may not accurately mimic a three-dimensional (3D) in vivo environment where cancer cells reside, because the 2D environment does not allow for areas of hypoxia, heterogeneous cell populations, varying cell proliferation zones, ECM influences, soluble signal gradients, and/or differential nutrient and metabolic waste transport.

3D cell-culture models may be utilized to overcome many limitations of 2D cell-culture models because 3D cell-culture models more closely mimic the features and environments of complex in vivo conditions. For example, studies have shown that tumor cells of specific cell lines, evaluated using 3D cell-culture models, are less sensitive to anti-cancer agents than when the same tumor cells are cultured using 2D cell-culture models.

One way to build 3D cell-culture models is to form 3D scaffolds out of porous polymeric and/or biologic materials. At least one study determined an optimal pore size (e.g., pore diameter) for 3D scaffolds of cell-culture models of approximately 100-400 μm. See Han, et. al, “Effect of Pore Size on Cell Behavior Using Melt Electrowritten Scaffolds,” Frontiers in Bioengineering and Biotechnology, Volume 9, Article 629270, July 2021. This result was based on based on achieving good cell nutrition, cell-growth space, and cell-scaffold interaction (the result depended on the specific cell lines).

Consistently controlling the size and shape of pores to achieve repeatable results requires a high precision manufacturing method. For example, to consistently create pores with diameters of 100 μm typically requires a manufacturing method having sub-20 μm resolution, such as micro 3D printing. In particular, projection micro stereolithography (PuSL) and two-photon polymerization are micro 3D printing techniques are capable of consistently creating complex 3D structures. Further, these printing techniques can create 3D structures from biocompatible and/or biodegradable polymers, such as poly-ethylene glycol (PEG) and poly lactic acid (PLA). After polymerization, these polymers may be hard or soft.

Cell cultures can be described in terms of reaction kinetics. In steady state, the process of cells consuming metabolites (e.g., cell nutrients) is often described by the Michaelis-Menten equation:

$$v = \frac{{V}_{max} [ S ]}{{K}_{M} + [ S ]} ,$$

where v is the velocity of the reaction, Vₘₐₓ is the maximum uptake rate of metabolites, K_{M} is the metabolite concentration when the uptake rate is half of the maximum (the Michaelis constant), and [S] is the concentration of metabolites. In Michaelis-Menten kinetics, the consumption behavior follows first order kinetics at low concentration. This means the consumption rate is proportional to the concentration. As the concentration of the metabolite increases, the consumption behavior will gradually become zero order kinetics. This means the consumption rate is near or equal to the maximum velocity and is independent of metabolite concentration. This is because the cells eventually become saturated and therefore their intake of metabolites reaches a plateau. Several embodiments of the bioreactor disclosed herein deliver metabolites to cells within the bioreactor using capillary tubes, such that even when the cells are densely packed, the cells are able to achieve a maximum uptake of metabolites for healthy functioning.

In an in vivo environment, when the cell concentration is on the order of 10¹⁰ cells/ml, the cells stay less than 100 μm from blood capillaries. To supply metabolites to such a high-density cell cluster, a high density of nutrient transport passages (capillaries) is required. The embodiments described herein disclose a perfusion-based 3D bioreactor for 3D cell cultures with a capillary system to promote 3D cell-to-cell and cell-to-ECM interactions as well as to promote transportation of metabolites and metabolic waste. Several embodiments are well suited for high cell-density 3D cell cultures. Such a perfusion-based 3D bioreactor can more accurately model in vivo biological conditions and cellular behaviors, which can aid in improving drug development processes. Some or all of the structures of the perfusion-based 3D bioreactor may be created via PμSL micro 3D printing and two-photon polymerization techniques.

### SUMMARY

The disclosed embodiments provide for a perfusion-based 3D bioreactor and a method of using the same. More specifically, the disclosed embodiments provide for a perfusion-based 3D bioreactor and a method of use that can more accurately model in vivo biological conditions and cellular behaviors for studying cell biology and tissue engineering, for example, high-density cell-to-cell interaction, high-density cell-to-ECM interaction, high-density cell-to-drug response, bio-material development, and new drug development. Some or all of the structures of the perfusion-based 3D bioreactor may be created via PμSL micro 3D printing and two-photon polymerization techniques.

Some embodiments of the bioreactor comprise a cavity on an upper surface of the bioreactor separating an inlet from a first return compartment and an outlet from the first return compartment; at least one first capillary artery tube crossing through the cavity and in fluidic communication with the inlet and the first return compartment; and at least one first capillary vein tube crossing through the cavity and in fluidic communication with the outlet and the first return compartment.

The bioreactor may further comprise an inlet compartment between the inlet and the at least one first artery tube; and an outlet compartment between the outlet and the at least one first vein tube; wherein at least a portion of the outlet compartment is gravitationally above the inlet compartment.

The bioreactor may further comprise an opening defined on a bottom surface of the bioreactor adjoining the cavity.

The bioreactor may further comprise at least one second capillary artery tube crossing through the cavity and in fluidic communication with the inlet compartment and a second return compartment adjacent to the first return compartment; and at least one second capillary vein tube crossing through the cavity and in fluidic communication with the outlet compartment and the second return compartment.

The bioreactor may further comprise at least one substantially vertical perforated support rib that structurally supports at least an individual one of the at least one first artery tube, the at least one first vein tube, the at least one second artery tube, and the at least one second vein tube.

In some embodiments, a plurality of the first artery tubes are arranged in a first arterial column and a plurality of first vein tubes are arranged in a first venous column horizontally adjacent to the first artery column; and a plurality of second artery tubes are arranged in a second arterial column and a plurality of second vein tubes are arranged in a second venous column horizontally adjacent to the second artery column; wherein either the first arterial column is horizontally adjacent to the second venous column or the first venous column is horizontally adjacent to the second arterial column.

In some embodiments, at least an individual one of the plurality of the first artery tubes, the plurality of the first vein tubes, the plurality of the second artery tubes, and the plurality of the second vein tubes includes micro holes.

In some embodiments, at least a portion of the bioreactor is 3D printed using projection micro stereolithography and two-photon polymerization.

In some embodiments, cells may be cultured by immersing the bioreactor into a culture-media bath contained within a basin; seeding the cavity with cells, incubating the cells, and pumping an input culture media into the inlet.

In some embodiments, an output culture media discharged from the outlet may be captured; at least some of the output culture media may be processed with a waste treatment device to obtain a treated output culture media; and at least some of the treated output culture media may be returned to the inlet.

The basin may further comprise a transparent window on a bottom surface thereof that overlaps with the opening of the bioreactor for viewing of the cavity and the cells therein.

The basin may further comprise an input port fluidically coupled to the inlet; and an output port fluidically coupled to the outlet.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings.

FIG. 1A shows a perspective view of an exemplary bioreactor from above and FIG. 1B shows a perspective view of the exemplary bioreactor from below.

FIG. 2 shows an exemplary arrangement of capillary tubes of a bioreactor.

FIGS. 3A-3B show one or more return compartments of the exemplary bioreactor, where FIG. 3A is a cross-section taken along line X-X in FIG. 1A and FIG. 3B is a cross-section taken along line A-A in FIG. 3A.

FIGS. 4A-4C show inlet and/or outlet compartments of the exemplary bioreactor, where FIG. 4A is a cross-section taken along line B-B in FIG. 4C, FIG. 4B is a cross-section taken along line C-C of FIG. 4A, and FIG. 4C is a cross-section taken along line D-D in FIG. 4A.

FIG. 5 shows an exemplary system for studying cell biology and/or tissue engineering that utilizes the exemplary bioreactor.

### DETAILED DESCRIPTION

The present disclosure may be more readily understood by reference to the following detailed description and the accompanying drawings, which form a part of this disclosure. This disclosure is not limited to the specific devices, methods, conditions, or parameters described and/or shown herein, and the terminology used herein is for the purpose of describing particular embodiments by way of example only and is not intended to be limiting of disclosed embodiments or inventions. For example, “left,” “right,” “clockwise,” and “counterclockwise” may be used as specific examples of generally opposite lateral or rotational directions, respectively. Also, as used in the specification and including the appended claims, the singular forms “a,” “an,” and “the” include the plural, and reference to a particular numerical value includes at least that particular value, unless the context clearly dictates otherwise.

The following numerals are used to describe various features of the embodiments.
10 bioreactor
20 cavity
30 opening
40 inlet
50 outlet
100 capillary tubes
102 artery capillary
104 vein capillary
110 support ribs
120 perforation holes
130 returns
200 return compartment
210 inlet compartment
220 outlet compartment
300 inlet pump
310 outlet reservoir
320 inlet seal
330 outlet seal
400 basin
410 window
420 culture-media bath
500 microscope

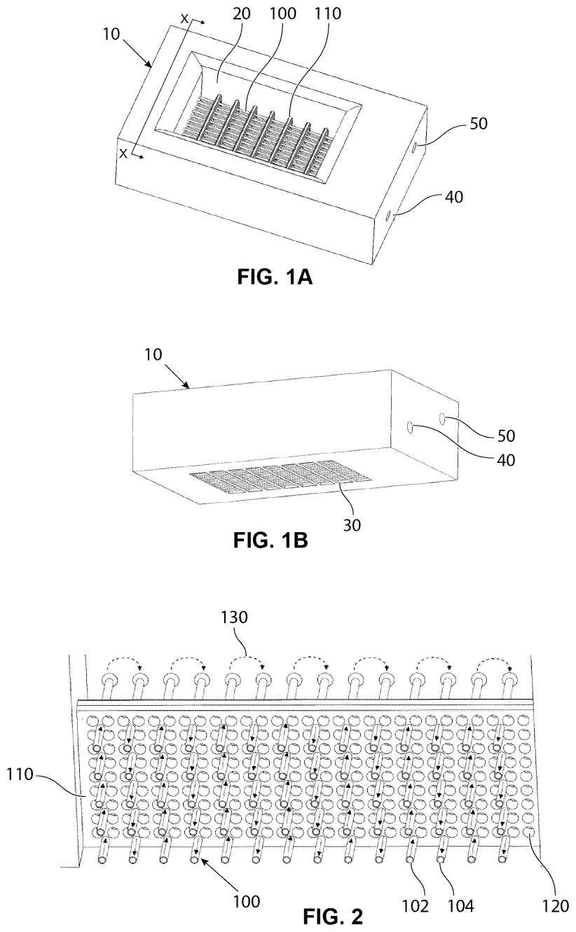

FIGS. 1A-1B show an embodiment of an exemplary perfusion-based 3D bioreactor 10 having a bowl-shaped cavity 20 defined on a top surface of the bioreactor 10, such that a flared top rim of the cavity 20 is adjacent to the top surface of the bioreactor 10. An opening 30 is defined on a bottom surface of the bioreactor 10 opposite the top surface and extending upward to a bottom of the cavity 20 such that at least a portion of the cavity 20 and the opening 30 bore vertically through the bioreactor 10. In some embodiments, the cavity size may be approximately 10 mm long, 6 mm wide, and 5 mm deep. As described herein, a longitudinal direction of the bioreactor 10 may be parallel to the length of the cavity 20 and a lateral direction of the bioreactor 10 may be parallel to the width of the cavity 20. The bioreactor 10 includes an inlet 40 and an outlet 50 each adapted for receiving or discharging fluid, such as cell-culture media, and for transporting the same to or from the cavity 20, respectively. In some embodiments, the inlet 40 and outlet 50 are disposed on a same longitudinal end of the bioreactor 10. The terms “cell-culture media” and “culture media” may be used interchangeably herein.

Near the bottom of the cavity 20, adjacent to the opening 30, is a plurality of capillary tubes 100 arranged to longitudinally cross the cavity 20. To be clear, the capillary tubes 100 are disposed within the cavity 20 and they extend from one side of the cavity 20 to an opposite side thereof. In some embodiments, the capillary tubes 100 are oriented parallel to each other. In some embodiments, there are five layers (e.g., rows) of capillary tubes 100, and each layer has 14 individual capillary tubes, for a total of 70 capillary tubes 100. In some embodiments, each capillary tube has an outer diameter of 100 μm and an inner diameter of 80 μm; the spacing between adjacent outer surfaces of capillary tubes 100 is 0.4 mm; and an equivalent transverse cross-sectional density of capillaries is 4 capillaries/mm². Other capillary arrangements, orientations, dimensions, and densities may be utilized. For example: (i) some or all of the capillary tubes 100 may be arranged to extend longitudinally, laterally, and/or diagonally across the cavity 20; (ii) some or all of the capillary tubes 100 may be oriented parallel and/or at various angles to each other; (iii) some or all of the capillary tubes 100 may have varying inner diameters, outer diameters, and wall thicknesses; (iv) some or all of the capillary tubes 100 may have varying spacings therebetween; and (v) the transverse cross-sectional density of capillaries may be approximately 3 to 30 capillaries/mm². Arranging capillary tubes 100 into columns may be beneficial for observing cells and/or tissues therebetween, as viewed from below through the opening 30 via microscopy as shown in FIG. 5, because lower capillary tubes 100 (closer to the opening 30) may not occlude the horizontal spaces between higher capillary tubes 100 (further away from the opening 30).

One or more support ribs 110 may be disposed within the cavity 20 for supporting the capillary tubes 100 extending therethrough. The number of support ribs 110 may depend on the outer diameters, wall thicknesses, and materials of the capillary tubes 100. For example, an embodiment where the capillary tubes 100 extend longitudinally across a 10 mm long cavity and each capillary tube 100 has an outer diameter of 100 μm, a wall thickness of 10 μm, and is constructed from polyethylene glycol, seven equally spaced support ribs 110 may be required to adequately support the capillary tubes 100. In some embodiments consisting of a low quantity of capillary tubes 100 (e.g., two capillary tubes 100) or a low density of capillary tubes 100 (e.g., where the spacings between adjacent capillary tubes 100 is approximately an order of magnitude greater than the outer diameters of the capillary tubes 100), individual vertical and/or horizontal support members may be utilized to structurally support the capillary tubes in lieu of perforated support ribs 110.

In some embodiments, the bioreactor 10 is formed or printed from class I or higher biocompatible material such as polyethylene glycol (PEG, molecular weight 575). Some materials may require a surface treatment to promote cell adhesion, which may be important for cell-culture experiments that need model cells to attach to and proliferate on a surface of the bioreactor 10. A poly-L-lysine solution may be used to coat one or more surfaces of the bioreactor 10, for example, one or more surfaces of the cavity 20.

FIG. 2 shows an exemplary arrangement of capillary tubes 100 comprising horizontal rows (e.g., layers) and vertical columns. The capillary tubes 100 intersect a support rib 110 that provides structural support therefor. Each support rib 110 includes a plurality of perforation holes 120 adapted to allow cells on either side of the support rib 110 to interact with each other, and also to allow cells to grow therein. In some embodiments, each perforation hole 120 has a diameter of 200 μm. Although FIG. 2 shows the perforation holes as uniformly spaced circular bores through the support rib 110, other spacings and shapes may be utilized.

Certain adjacent capillary tubes 100 fluidically communicate via a return 130. As shown in FIG. 2, horizontally adjacent capillary tubes 100 fluidically communicate via a return 130, such that a first column of capillary tubes 100 fluidically communicates with a second column, a third column fluidically communicates with a fourth column, and so on in an alternating manner. Thus, the flow of fluid in adjacent columns of capillary tubes 100 is in opposite directions. Capillary tubes 100 carrying fluid in a first direction (into a return 130) are called artery capillaries 102 (or artery tubes) herein, and capillary tubes 100 carrying fluid in a second direction (out of a return 130) are called vein capillaries 104 (or vein tubes) herein. Artery capillaries 102 serve to transport fresh culture media and to deliver the same to cells within the cavity 20, while vein capillaries serve to transport metabolic waste and remove the same from the cells with the cavity 20. In an alternate embodiment, vertically adjacent capillary tubes 100 fluidically communicate via a return 130, such that a first row of capillary tubes 100 fluidically communicates with a second row, a third row fluidically communicates with a fourth row, and so on in an alternating manner.

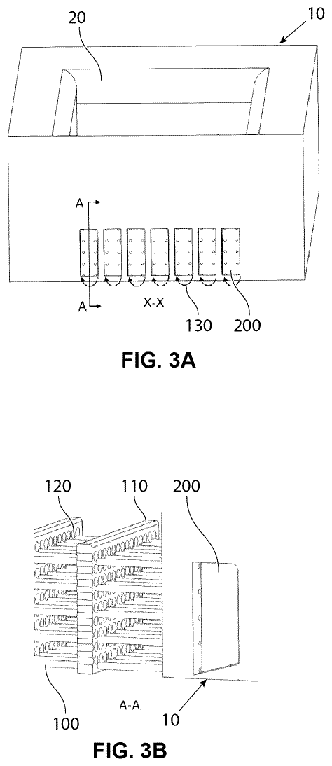

FIG. 3A is a cross-section of a bioreactor 10 showing a plurality of return compartments 200, each of which comprises a plurality of returns 130 between an artery capillary 120 of an i^{th} column and an adjacent vein capillary 104 of an (i+1)^{th} column. FIG. 3B shows a cross-section of a single return compartment 200. In some embodiments, there are seven return compartments 200 each comprising seven returns 130 each between an artery capillary 102 and an adjacent vein capillary 104. In an alternate embodiment, each return compartment 200 may consist of a single return 130 between a single artery capillary 120 and a single adjacent vein capillary 104.

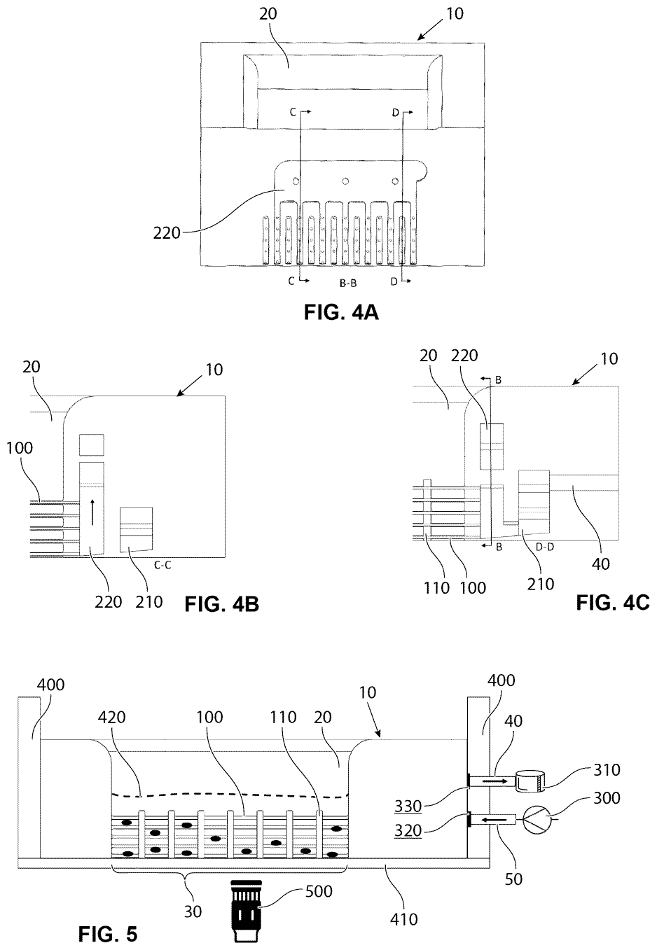

FIGS. 4A-4C show various ports and compartments of a bioreactor 10 for receiving, discharging, distributing, consolidating, and/or transporting of fluid such as solutions of fresh culture media and metabolic waste. The artery capillaries 102 are in fluidic communication with the inlet 40 via an inlet compartment 210. The inlet 40 is adapted to receive fresh culture media from an external source and to deliver the same to the inlet compartment 210. As shown in FIG. 4A and FIG. 4C, the inlet compartment 210 distributes fluid, for example fresh culture media, to all artery capillaries 102. The vein capillaries 104 fluidically communicate with the outlet 50 via an outlet compartment 220. The outlet 50 is adapted to discharge metabolic waste delivered from the outlet compartment 220. As shown in FIG. 4A and FIG. 4B, the outlet compartment 220 consolidates fluid, for example metabolic waste, from all vein capillaries 104.

Diffusion of solutes across a membrane, such as a wall of a capillary tube 100, is proportional to the concentration gradient of solutes across the membrane. Thus, the wall thicknesses and/or materials of the capillary tubes 100 are selected to allow for an appropriate rate of diffusion of certain solutes into and out of the capillary tubes 100, including metabolites and metabolic waste. For example, if the concentration of metabolic waste in the cavity 20 becomes too high (e.g., approximately 100 μmol/L), the health of the cells in the cavity 20 may suffer. Metabolic waste may include toxins such as nitrogen compounds. Similarly, if the concentration of metabolites in the cavity becomes too low, then cells may fail to proliferate and/or grow, or cells may starve and/or die. It is therefore important to supply the cavity 20 with sufficient metabolites and to continually remove metabolic waste.

In some embodiments, diffusion through the wall of the capillary tubes 100 (for one or both of metabolites and metabolic waste) may be increased by adding micro holes to the walls of the capillary tubes 100. In some embodiments, micro holes having diameters of approximately 5-15 μm can be added to the walls of the capillary tubes 100 to significantly increase the rate(s) of diffusion. In some embodiments, the diameters and/or density (spacings) of micro holes added to artery capillaries 102 may be different than the diameters and/or density (spacings) of micro holes added to vein capillaries 104. In some embodiments, micro holes may be added only to artery capillaries 102 or only to vein capillaries 104, or only to a select portion thereof.

As culture media is transported within the capillaries 100, metabolites may diffuse out through the walls thereof, where they may be consumed by cells within the cavity 20. Accordingly, cells that consume metabolites may produce and release metabolic waste, which may diffuse in through the walls of the capillaries 100, where they may be transported thereby. The flow directions of culture media in the capillary tubes 100 is indicated by the arrows shown in FIG. 2; artery capillaries 102 transport fresh culture media from the inlet compartment 210 to a return compartment 200, and vein capillaries 104 transport culture media from the return compartments 200 to the outlet compartment 220. Thus, metabolic waste that diffuses into a vein capillary 104 is transported therein to the outlet compartment 220, while metabolic waste that diffuses into an artery capillary 102 is transported to a return compartment 200 where it will enter a vein capillary 104 and subsequently be transported to the outlet compartment 220. In some embodiments, the flow rate within the capillary tubes 100 is on the order of several millimeters per second.

FIGS. 4A-4C show the outlet compartment 220 having a portion that is gravitationally above a highest portion of the inlet compartment. This configuration helps to remove air or other gases that may be introduced into the capillary tubes 100. Accordingly, it may be advantageous for the outlet 50 to connect to the outlet compartment 220 at or near a highest portion thereof.

FIG. 5 shows an exemplary system for studying cell biology and/or tissue engineering comprising a bioreactor 10 disposed within a basin 400 having a transparent window 410 on a bottom surface thereof. The window can be made from any suitable transparent material, for example quartz or clear plastic, and can have any suitable thickness, for example 0.5 to 1.0 mm. At least a portion of the window 410 should overlap with a portion of the opening 30 such that it may be possible to visually observe a portion of the cavity 20 from below. A microscope 500 may be utilized for such observation. The basin 400 may contain a culture-media bath 420, such that the capillary tubes 100 may be immersed or submerged therein.

The inlet 40 may be coupled to an input port of the basin 400 that is in fluidic communication with an inlet pump 300 for supplying fresh culture media. The inlet pump 300 may draw the fresh culture media from a fresh-storage reservoir (not illustrated). An inlet seal 320, for example an O-ring, may be disposed between the inlet 40 and the input port to prevent leakage. The outlet 50 may be coupled to an output port of the basin 400 that is in fluidic communication with an outlet reservoir 310 for receiving metabolic waste. An outlet seal 330, for example an O-ring, may be disposed between the outlet 50 and the output port to prevent leakage. Alternatively, the inlet pump 300 may directly feed into the inlet 40 and the outlet 50 may directly feed into the outlet reservoir 310.

In some embodiments, the outlet reservoir 310 may be fluidically coupled to a waste treatment device (not illustrated) for separating metabolic waste from metabolites, and there may be a means for transporting the recovered metabolites to the inlet 40 for recirculation. For example, the waste treatment device may transport the recovered metabolites to a recovered-storage reservoir (not illustrated) from which recovered metabolites may be drawn via pumping or by gravity. In the case of pumping, the inlet pump 300 may transport the recovered and/or reprocessed metabolites from the recovered-storage reservoir to the inlet 40, or alternatively, a second pump may transport the recovered and/or reprocessed metabolites to the inlet 40 or to the fresh-storage reservoir.

The bioreactor 10 may be seeded with cells by any suitable manner. For example, seed cells may be suspended in a culture media and pipetted into the cavity 20 manually or in an automated manner. Unfortunately, gravity may cause the initial seed cells to collect at the bottom of the cavity 20, adjacent to the window 410, instead of attaching to the capillary tubes 100. However, as more seed cells are pipetted into the cavity 20, at least some seed cells are expected to attach to the capillary tubes 100. Alternatively, seed cells may be suspended in a hydrogel pre-solution capable of gelation or polymerization, for example upon exposure to air or ultraviolet light. Gelation or polymerization causes the suspended seed cells to become encapsulated by the hydrogel, which prevents them from sinking to the bottom of the cavity 20 and collecting on the window 410. To be clear, the hydrogel pre-solution may be exposed to air or ultraviolet light after exiting the pipette and before entering the culture-media bath 420 within the cavity 20.

Once the bioreactor 10 is seeded with cells, it may be placed into an incubator (not illustrated) where environmental conditions may be controlled to promote (or discourage) cell growth and/or proliferation. Controlled environmental conditions may include temperature, humidity, and CO₂ concentration.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

1. A perfusion-based 3D bioreactor, comprising:
a cavity on an upper surface of the bioreactor separating an inlet from a first return compartment and an outlet from the first return compartment;
at least one first capillary artery tube crossing through the cavity and in fluidic communication with the inlet and the first return compartment; and
at least one first capillary vein tube crossing through the cavity and in fluidic communication with the outlet and the first return compartment.

2. The bioreactor of claim 1 further comprising:
an inlet compartment between the inlet and the at least one first capillary artery tube; and
an outlet compartment between the outlet and the at least one first capillary vein tube; wherein
at least a portion of the outlet compartment is gravitationally above the inlet compartment.

3. The bioreactor of claim 2 further comprising an opening defined on a bottom surface of the bioreactor adjoining the cavity.

4. The bioreactor of claim 3 further comprising:
at least one second capillary artery tube crossing through the cavity and in fluidic communication with the inlet compartment and a second return compartment adjacent to the first return compartment; and
at least one second capillary vein tube crossing through the cavity and in fluidic communication with the outlet compartment and the second return compartment.

5. The bioreactor of claim 4 further comprising at least one substantially vertical perforated support rib that structurally supports at least an individual one of; the at least one first capillary artery tube, the at least one first capillary vein tube, the at least one second capillary artery tube, or the at least one second capillary vein tube.

6. The bioreactor of claim 5 wherein:
a plurality of the at least one first capillary artery tubes is arranged in a first arterial column and a plurality of at least one first capillary vein tubes is arranged in a first venous column horizontally adjacent to the first artery column; and
a plurality of the at least one second capillary artery tubes is arranged in a second arterial column and a plurality of the at least one second capillary vein tubes is arranged in a second venous column horizontally adjacent to the second artery column; wherein
either the first arterial column is horizontally adjacent to the second venous column or the first venous column is horizontally adjacent to the second arterial column.

7. The bioreactor of claim 6 wherein at least an individual one of; the plurality of the at least one first capillary artery tubes, the plurality of the at least one first capillary vein tubes, the plurality of the at least one second capillary artery tubes, or the plurality of the at least one second capillary vein tube includes micro holes.

8. The bioreactor of claim 7 wherein at least a portion of the bioreactor is 3D printed using projection micro stereolithography and two-photon polymerization.

9. The bioreactor of claim 7 wherein a density of all capillary tubes is 3 to 30 capillaries/mm².

10. A method of culturing cells, comprising:
immersing a 3D perfusion bioreactor into a culture-media bath contained within a basin, the bioreactor comprising:
a cavity on an upper surface of the bioreactor separating an inlet from a first return compartment and an outlet from the first return compartment,
at least one first capillary artery tube crossing through the cavity and in fluidic communication with the inlet and the first return compartment, and
at least one first capillary vein tube crossing through the cavity and in fluidic communication with the outlet and the first return compartment;
seeding the cavity with cells;
incubating the cells; and
pumping an input culture media into the inlet.

11. The method of claim 10 further comprising:
capturing an output culture media discharged from the outlet;
processing at least some of the output culture media with a waste treatment device to obtain a treated output culture media; and
returning at least some of the treated output culture media to the inlet.

12. The method of claim 11 wherein:
the bioreactor further comprises an opening defined on a bottom surface thereof adjoining the cavity; and
the basin further comprises a transparent window on a bottom surface thereof.

13. The method of claim 12 wherein the basin further comprises:
an input port fluidically coupled to the inlet; and
an output port fluidically coupled to the outlet.

14. The method of claim 13 wherein the bioreactor further comprises:
an inlet compartment between the inlet and the at least one first capillary artery tube; and
an outlet compartment between the outlet and the at least one first capillary vein tube; wherein
at least a portion of the outlet compartment is gravitationally above the inlet compartment.

15. The method of claim 14 wherein the bioreactor further comprises:
at least one second capillary artery tube crossing through the cavity and in fluidic communication with the inlet compartment and a second return compartment adjacent to the first return compartment; and
at least one second capillary vein tube crossing through the cavity and in fluidic communication with the outlet compartment and the second return compartment.

16. The method of claim 15 wherein the bioreactor further comprises at least one substantially vertical perforated support rib that structurally supports at least an individual one of the at least one first capillary artery tube, the at least one first capillary vein tube, the at least one second capillary artery tube, or the at least one second capillary vein tube.

17. The method of claim 16 wherein:
a plurality of the at least one first capillary artery tubes is arranged in a first arterial column and a plurality of the at least one first capillary vein tubes is arranged in a first venous column horizontally adjacent to the first artery column; and
a plurality of the at least one second capillary artery tubes is arranged in a second arterial column and a plurality of the at least one second capillary vein tubes is arranged in a second venous column horizontally adjacent to the second artery column; wherein
either the first arterial column is horizontally adjacent to the second venous column or the first venous column is horizontally adjacent to the second arterial column.

18. The method of claim 17 wherein at least an individual one of; the plurality of the at least one first artery tubes, the plurality of the at least one first vein tubes, the plurality of the at least one second artery tubes, or the plurality of the at least one second vein tubes includes micro holes.

19. The method of claim 18 wherein at least a portion of the bioreactor is 3D printed using projection micro stereolithography and two-photon polymerization.

20. The method of claim 18 wherein a density of all capillary tubes is 3 to 30 capillaries/mm².
